(19) United States
(12) Patent Application Publication
(10) Pub. No.: US 2018/0193321 A1
(43) Pub. Date: Jul. 12, 2018

(54) INDOLOTRYPTOLINE ANTICANCER AGENTS

(71) Applicants: Sean BRADY, New York, NY (US); Daniel MONTIEL, New York, NY (US)

(72) Inventors: Sean BRADY, New York, NY (US); Daniel MONTIEL, New York, NY (US)

(73) Assignee: The Rockefeller University, New York, NY (US)

(21) Appl. No.: 15/741,052

(22) PCT Filed: Jul. 1, 2016
(86) PCT No.: PCT/US2016/040793

§ 371 Date: Dec. 29, 2017

Related U.S. Application Data
(60) Provisional application No. 62/187,873, filed on Jul. 2, 2015.

(51) Int. Cl. A61K 31/437; A61P 35/00

(52) U.S. Cl. CPC A61K 31/437; A61P 35/00

(57) ABSTRACT

Indolotryptoline compounds of the general structure:

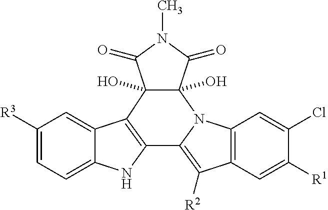

are disclosed. In these compounds R¹ is chosen from hydrogen, halogen and OH; R² is (C₁-C₄)alkoxy; and R³ is chosen from hydrogen and halogen The compounds are useful for treating cancer.

### CROSS-REFERENCE TO RELATED APPLICATION

This application claims priority benefit of U.S. Provisional Patent Application No. 62/187,873, filed Jul. 2, 2015, the disclosure of which is hereby incorporated by reference herein in its entirety.

### FEDERALLY SPONSORED RESEARCH

This invention was made with government support under NIH GM 077516 awarded by the National Institutes of Health. The government has certain rights in the invention.

### FIELD OF THE INVENTION

The invention relates to compounds in the indolotryptoline family that inhibit the growth of neoplastic cells. These compounds are useful to treat various cancers.

### BACKGROUND OF THE INVENTION

Tryptophan dimers (or bisindoles) are a structurally and functionally diverse class of natural products. The best studied of these are the indolocarbazoles staurosporine and rebeccamycin, which are kinase and topoisomerase inhibitors, respectively. One very potent, but to date, rarely encountered and therefore underexplored family of tryptophan dimers is the indolotryptolines. Indolotryptolines contain a core tri-cyclic tryptoline ring fused to an indole. The two naturally occurring indolotryptolines that have been characterized in fermentation based natural product discovery programs are cladoniamide and BE-54017. Both exhibit potent human cell cytotoxicity.

### SUMMARY OF THE INVENTION

In one aspect, the present disclosure provides compounds of Formula I:

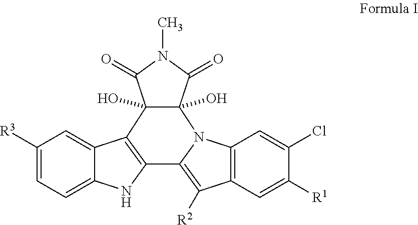

wherein

R¹ is chosen from hydrogen, halogen and OH;

R² is (C₁-C₄)alkoxy; and

R³ is chosen from hydrogen and halogen.

In some embodiments, R³ is chlorine. In some embodiments, R² is methoxy. In some embodiments, R¹ is hydrogen or OH. In some embodiments, R¹ is hydrogen or OH and R² is methoxy. In some embodiments R¹ is hydrogen or OH and R² is methoxy and R³ is chlorine.

In another aspect, the present disclosure provides a pharmaceutically acceptable salt or solvate of a compound having the structure of Formula I:

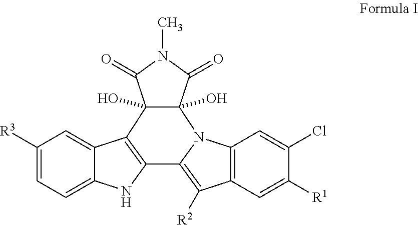

wherein

R¹ is chosen from hydrogen, halogen and OH;

R² is (C₁-C₄)alkoxy; and

R³ is chosen from hydrogen and halogen.

In some embodiments, R³ is chlorine. In some embodiments, R² is methoxy. In some embodiments, R¹ is hydrogen or OH. In some embodiments, R¹ is hydrogen or OH and R² is methoxy. In some embodiments R¹ is hydrogen or OH and R² is methoxy and R³ is chlorine.

In another aspect, the present disclosure provides methods for treating cancer comprising exposing a cell to a compound having the structure of Formula I:

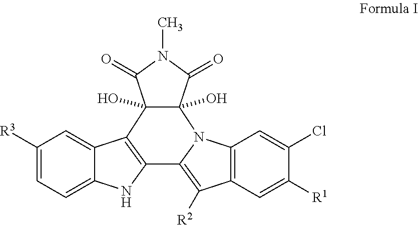

wherein

R¹ is chosen from hydrogen, halogen and OH;

R² is (C₁-C₄)alkoxy; and

R³ is chosen from hydrogen and halogen.

In some embodiments, R³ is chlorine. In some embodiments, R² is methoxy. In some embodiments, R¹ is hydrogen or OH. In some embodiments, R¹ is hydrogen or OH and R² is methoxy. In some embodiments R¹ is hydrogen or OH and R² is methoxy and R³ is chlorine.

In some embodiments, the cancer is an EGFR-overexpressing cancer.

In some embodiments, the cancer is a cancer selected from head and neck, ovarian, cervical, bladder, renal and oesophageal cancers, non-small cell lung cancer, bronchoalveolar carcinoma, gastric, breast, endometrial and colorectal cancers.

In some embodiments, the cancer is a cancer selected from non-small cell lung cancer, refractory breast cancer, renal cancer and colon cancer.

In another aspect, the invention relates to pharmaceutical compositions comprising a pharmaceutically acceptable carrier and a compound described herein.

### DETAILED DESCRIPTION OF THE INVENTION

The practice of the present invention will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, et al., Molecular Cloning: A Laboratory Manual (3rd Edition, 2000); DNA Cloning: A Practical Approach, vol. I & II (D. Glover, ed.); Oligonucleotide Synthesis (N. Gait, ed., 1984); Oligonucleotide Synthesis: Methods and Applications (P. Herdewijn, ed., 2004); Nucleic Acid Hybridization (B. Hames & S. Higgins, eds., 1985); Nucleic Acid Hybridization: Modern Applications (Buzdin and Lukyanov, eds., 2009); Transcription and Translation (B. Hames & S. Higgins, eds., 1984); Animal Cell Culture (R. Freshney, ed., 1986); Freshney, R.I. (2005) Culture of Animal Cells, a Manual of Basic Technique, 5th Ed. Hoboken N.J., John Wiley & Sons; B. Perbal, A Practical Guide to Molecular Cloning (3rd Edition 2010); Farrell, R., RNA Methodologies: A Laboratory Guide for Isolation and Characterization (3rd Edition 2005). Poly(ethylene glycol), Chemistry and Biological Applications, ACS, Washington, 1997; Veronese, F., and J. M. Harris, Eds., Peptide and protein PEGylation, Advanced Drug Delivery Reviews, 54(4) 453-609 (2002); Zalipsky, S., et al., “Use of functionalized Poly(Ethylene Glycols) for modification of polypeptides” in Polyethylene Glycol Chemistry: Biotechnical and Biomedical Applications. The publications discussed above are provided solely for their disclosure before the filing date of the present application. Nothing herein is to be construed as an admission that the invention is not entitled to antedate such disclosure by virtue of prior invention.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

The use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Throughout this specification the terms and substituents retain their definitions.

Unless otherwise stated or depicted, structures depicted herein are also meant to include all stereoisomeric (e.g., enantiomeric, diastereomeric, and cis-trans isomeric) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and cis-trans isomeric (or conformational) mixtures of the present compounds are within the scope of the invention. Unless otherwise stated, all tautomeric forms of the compounds of the invention are within the scope of the invention.

The graphic representations of racemic, ambiscalemic and scalemic or enantiomerically pure compounds used herein are taken from Maehr J. Chem. Ed. 62, 114-120 (1985): solid and broken wedges are used to denote the absolute configuration of a chiral element; wavy lines indicate disavowal of any stereochemical implication which the bond it represents could generate; solid and broken bold lines are geometric descriptors indicating the relative configuration shown but denoting racemic character; and wedge outlines and dotted or broken lines denote enantiomerically pure compounds of indeterminate absolute configuration. Thus, the formula X is intended to encompass both of the pure cis enantiomers of that pair:

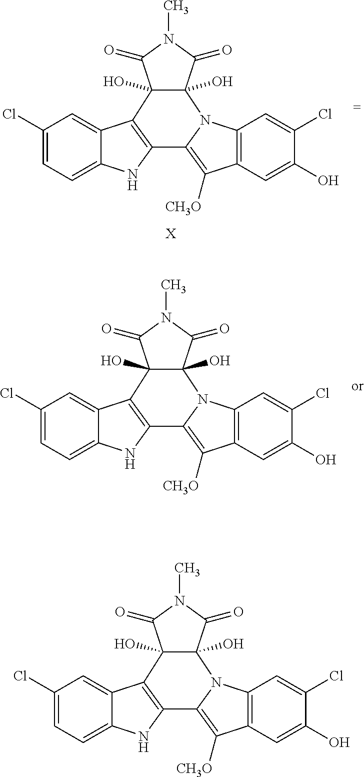

### Overview

The chemical diversity encoded by natural microbial communities has been significantly underexplored due to limitations associated with the inability to culture the majority of environmental bacteria and the silencing of biosynthetic pathways under laboratory conditions. Soils contain thousands of unique bacterial species, which potentially harbor tens of thousands of functionally unexplored natural product biosynthetic gene clusters. With the development of metagenomic cloning methods, it is possible to use DNA extracted directly from soil (environmental DNA, eDNA) to construct libraries that capture the enormous biosynthetic diversity present in soil environments. These libraries provide a means of functionally examining unexplored soil biosynthetic gene clusters and are, therefore, resources for sequence-guided natural product discovery programs. Using these libraries, we have identified novel compounds with clinically relevant biological activities.

In its most basic aspects, the invention relates to compounds having the structure:

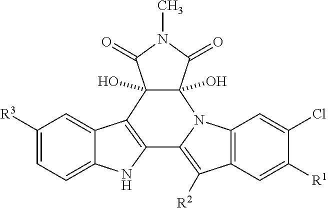

and to methods of using the compounds and pharmaceutical compositions containing the compounds.

In some embodiments, the invention relates to a pharmaceutically acceptable salt or solvate of such a compound having the structure:

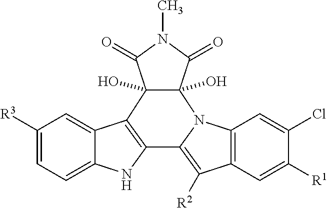

and to methods of using the pharmaceutically acceptable salt or solvate of the compound and pharmaceutical compositions containing the pharmaceutically acceptable salt or solvate of the compound.

In some embodiments, R³ is chlorine. In some embodiments, R² is methoxy. In some embodiments, R¹ is hydrogen or OH.

In some embodiments, the present disclosure provides administration of the compounds described herein to patients as the raw chemical. In some embodiments, the present disclosure provides administration of the compounds described herein as a pharmaceutical composition. According to a further aspect, the present invention provides a pharmaceutical composition comprising a compound disclosed herein together with one or more pharmaceutical carriers, diluent, or excipient and optionally one or more other therapeutic ingredients. The carrier(s), diluent(s) and excipient(s) must be “acceptable” (e.g., “pharmaceutically acceptable”) in the sense of being compatible with the other ingredients of the formulation and not deleterious to the recipient thereof.

Acceptable carriers diluents and excipients are known in the art and include, without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, l-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. For example, inorganic salts include, but are not limited to, ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Example organic bases used in certain embodiments include isopropylamine, diethylamine, ethanolamine, trimethylamine, dicyclohexylamine, choline and caffeine.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

As used herein, “patient” includes humans and mammals (e.g., mice, rats, pigs, cats, dogs, and horses). In many embodiments, patient are mammals, particularly primates, especially humans. In some embodiments, patient are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) patient mammals will be, for example, rodents (e.g., mice, rats, hamsters), rabbits, primates, or swine such as inbred pigs and the like.

“Treatment” or “treating,” as used herein, includes any desirable effect on the symptoms or pathology of a disease or condition, and may include even minimal changes or improvements in one or more measurable markers of the disease or condition being treated. “Treatment” or “treating” does not necessarily indicate complete eradication or cure of the disease or condition, or associated symptoms thereof. The patient receiving this treatment is any patient in need thereof. Exemplary markers of clinical improvement will be apparent to persons skilled in the art. In some embodiments, “Treating” and “Treatment” includes the delivering a compound (e.g., a pharmaceutically acceptable salt or solvate of a compound disclosed herein or a pharmaceutical compositions comprising a compound disclosed herein) to a patient to effect an outcome. The compound may be delivered by administration to the patient.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Exposing a cell” to a compound described herein includes without limitation contacting a cell in vitro or in vivo. In some embodiments, exposing a cell to a compound described herein is via administration of the compound or a pharmaceutically acceptable salt or solvate thereof, or a pharmaceutical composition comprising a compound disclosed herein to a patient.

The formulations for administration to patients include those suitable for oral, parenteral (including subcutaneous, intradermal, intramuscular, intravenous and intraarticular), rectal and topical (including dermal, buccal, sublingual and intraocular) administration. In most cases, parenteral administration will be preferred. The most suitable route may depend upon the condition and disorder of the recipient. The formulations may conveniently be presented in unit dosage form and may be prepared by any of the methods well known in the art of pharmacy.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste. A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

### EXAMPLES

### Example 1

### Evaluation of a Set of Constitutively Active Bi-Directional Promoter Cassettes for Activating Gene Clusters Using a Multiplexed Promoter Exchange Strategy

With the construction of a set of constitutively active bi-directional promoter cassettes and the ability to efficiently introduce these cassettes into gene clusters we sought to evaluate their utility for activating gene clusters using a multiplexed promoter exchange strategy. We initially explored this using well characterized natural product biosynthetic gene clusters known to encode for either the tryptophan dimer rebeccamycin (Reb) or the aromatic polyketide tetarimycin (Tam). The Reb cluster is natively transcriptionally active, while the Tam cluster is known to be transcriptionally silent. The Reb gene cluster used in this study was originally isolated from an Arizona soil metagenome and found to natively produce rebeccamycin when introduced into S. albus. The Reb gene cluster is predicted to contain three promoters: two promoters oriented in opposite directions between the rebG and rebO genes that were used to test our promoter cassettes and a third promoter upstream of the rebR gene. The bi-directional promoter site between the rebG and rebO genes was replaced with a TRP1 bidirectional promoter cassette while the uni-directional promoter in the upstream region of the rebR gene was replaced with a MET15 cassette, in which only one promoter was incorporated into the amplicon used for recombination (i.e., a uni-directional promoter cassette). This construct was then moved from yeast, through E. coli into S. albus for heterologous expression studies. HPLC analysis of organic extracts from cultures of S. albus transformed with the wild type Reb gene cluster or with the promoter exchanged Reb gene cluster showed no significant difference in the production of rebeccamycin, indicating that our promoter re-engineering tools are able to induce molecule production from transcriptionally silent natural product biosynthetic gene clusters.

The Tam gene cluster is an eDNA-derived type II (aromatic) polyketide synthase biosynthetic gene cluster that encodes for the antibiotics tetarimycin A and B. In S. albus, this gene cluster is transcriptionally silent unless tamI, the gene cluster specific SARP family positive regulator, is artificially up-regulated. The Tam gene cluster is predicted to contain eight biosynthetic operons driven by four promoter regions. We replaced all four promoter regions with synthetic promoter cassettes using two rounds of TAR. In the first yeast transformation, LEU2, MET15, TRP1 and HISS-based promoter cassettes were inserted in parallel into the Tam gene cluster using 40 bp homology arms. Promoter cassettes with 500 bp homology arms were then amplified from each re-engineered gene cluster. In the second round of TAR, LEU2, MET15 and TRP1-based promoter cassettes with 500 bp homology arms were simultaneously inserted into the Tam gene cluster harboring the HIS3 promoter cassette. The successful insertion of all four promoter cassettes into the Tam cluster was confirmed by genotyping re-factored gene clusters using PCR. The promoter re-engineered Tam cluster was transformed into E. coli S17 and conjugated into S. albus for heterologous expression studies. LC-MS analysis of culture broth extracts from S. albus transformed with either the promoter refactored Tam cluster or the wild type cluster activated through induced expression of the SARP regulatory element showed essentially identical levels of tetarimycin production, indicating that the complete promoter refactoring was able to replicate native levels of metabolite production by this gene cluster.

Silent gene clusters in need of activation increasingly appear in (meta)genomic DNA sequencing datasets. We used our promoter engineering method to activate a previously uncharacterized silent, and we believe naturally dead, eDNA-derived indolotryptoline gene cluster, which we refer to as the Lzr gene cluster, to produce a novel indolotryptoline metabolite with potent human cell cytotoxicity. Indolotryptolines contain a core tri-cyclic tryptoline ring fused to an indole. The two naturally occurring indolotryptolines that have been characterized in fermentation based natural product discovery programs, are cladoniamide and BE-54017. Both exhibit potent human cell cytotoxicity. In an effort to expand the observed natural diversity of indolotryptolines and potentially improve their therapeutic potential, we PCR screened eDNA cosmid libraries using degenerate primers targeting genes that encode for tryptophan dimerization enzymes. This led to the discovery of the Lzr gene cluster, which closely resembles the cladoniamide and BE-54017 gene clusters; however, it encodes tailoring enzymes (e.g., an extra halogenase and a cytochrome p450 oxidase) that are not used in the biosynthesis of any known indolotryptoline, suggesting that it would encode for a novel indolotryptoline congener. The Lzr gene cluster was recovered from a previously archived Arizona desert soil eDNA library on two overlapping eDNA cosmid clones (AZ25-292 and AZ25-153). The full-length Lzr gene cluster was re-assembled from these two cosmids using TAR and a pTARa-based pathway-specific E. coli:yeast:Streptomyces shuttle capture vector to yield the bacterial artificial chromosome (BAC) BAC-AZ25-292/153. This BAC was transferred into S.albus for heterologous expression studies, but this strain failed to produce any detectable clone-specific metabolite under all of the culture conditions we tested, indicating that the Lzr gene cluster is silent in S. albus. We used this silent cryptic gene cluster for testing our promoter replacement tools.

The outer edges of the Lzr gene cluster were defined based on comparisons to the BE-54017 and cladoniamide gene clusters and a BLAST analysis of genes surrounding the core indolotryptoline biosynthesis genes. The biosynthesis of indolotryptolines is well-characterized making it possible to predict the function of most genes in the Lzr gene cluster. The four key stages of indolotryptoline biosynthesis involve dimerization of oxo-tryptophan to form a chromopyrrolic acid; oxidative aryl-aryl coupling to form an indolocarbazole; ‘flipping’ of one of the indole rings to form a indolotryptoline; and tailoring to generate the final product. The Lzr gene cluster appears to contain seven transcriptional units controlled by three bi-directional and one uni-directional promoter regions. This cluster is conveniently organized such that successive activation of three bi-direction promoter regions P1, P2 and P3 were expected to drive the expression of genes required to achieve the first, second, and third stages in indolotryptoline biosynthesis, respectively. In a series of single cassette insertions we replaced each bidirectional Lzr promoter region with a synthetic promoter cassette. As expected, P1 and P1+P2 replacement constructs produced chromopyrrolic and indolocarbazole intermediates, respectively. The P1+P2+P3 replaced gene cluster, however, produced an indolocarbazole intermediate instead of the expected indolotryptoline intermediate. A close examination of lzrX1, the gene predicted to encode the oxidative enzyme that installs the C4c/C7a diol, suggested that a single base deletion had led to a truncated and likely non-functional lzrX1gene (i.e., pseudogene). Hence, the Lzr gene cluster appears to be not only silent but also dead due to the disruption of the lzrX1 gene.

Ideally, a gene cluster activation tool should therefore not only be able to awaken silent gene clusters through the replacement of promoters but also have the flexibility to “resuscitate” dead gene clusters through the exchange of pseudogenes with functional homologs found in closely related gene clusters. In an effort to resuscitate the Lzr cluster, we extended our promoter exchange method to allow for simultaneous insertion of both synthetic promoters and new genes into the gene cluster of interest. In this case the abeX1gene from the BE-54017 gene cluster, a full-length homolog of the lzrX1 pseudogene, and a promoter selection cassette were independently PCR amplified to produce amplicons with 20 bp overlaps. A second round of PCR was then carried out to link the resulting amplicons into a single cassette containing 40 bp Lzr cluster specific homology arms, two promoters, the full-length abeX1 oxidative gene and the LYS2 marker gene. This cassette was then used in a standard TAR promoter exchange reaction to replace both the disrupted lzrX1 gene and the P3 promoter region. Upon introduction of this cassette into the P3 site, the new P1+P2+P3 re-engineered gene cluster was found to confer to S. albus the ability to produce a new indolocarbazole, which we call lazarimide C, and a new indolotryptoline-based metabolite (compound 6), which we call lazarimide B.

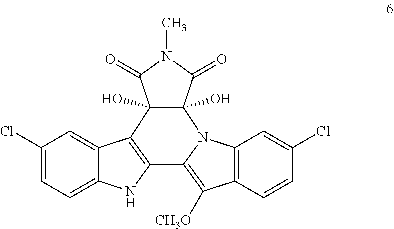

### Example 2

### Completion of the Re-Factoring of the Lzr Gene Cluster Via Replacement of P4 with a Uni-Directional Synthetic Promoter Cassette

To complete the re-factoring of the Lzr gene cluster, we replaced P4 with a uni-directional synthetic promoter cassette. Heterologous expression studies with this fully re-engineered Lzr gene cluster showed the presence of one additional major metabolite not seen in cultures of S. albus transformed with any previous re-engineered constructs. Compound 7, which we have given the trivial name lazarimide A, was purified from large-scale cultures of S. albus transformed with the completely re-engineered Lzr gene cluster, and its structure was solved using HRESIMS, and 1D and 2D NMR.

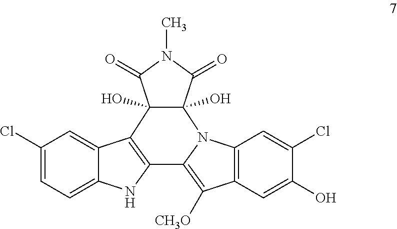

The general structure of the lazarimide series of metabolites was further confirmed with a crystal structure of the lazarimide intermediate lazarimide C. Lazarimide A (7) differs from cladoniamide and BE-54017 by both its halogenation pattern and the oxidation of the flipped indole moiety. Details of the biochemistry can be found in Montiel, Kang, Chang, Charlop-Powers and Brady, “Yeast homologous recombination-based promoter engineering for the activation of silent natural product biosynthetic gene clusters”, Proc.Nat.Acad. Sci. US, published Jul. 6, 2015.

### Example 3

### Measuring Compound Cytotoxicity

Because known indolotryptolines are potent human cell line toxins, lazarimide C, novel compounds 6 and 7, as well as cladoniamide A as a control, were tested for cytotoxicity against HCT-116 human colon carcinoma cancer cells. The IC₅₀s observed for cladoniamide A, lazarimide C, and compounds 6 and 7 were 40.4 nM, 25.8 μM, 11.6 nM and 8.4 nM, respectively.

The compounds were tested for antiproliferative activity in triplicate using the colon carcinoma cell line HCT-116 (ATCC; CCL-247) [See Brattain et al., “Heterogeneity of malignant cells from a human colonic carcinoma.” Cancer Res. 41(5):1751-1756(1981), incorporated herein by reference in its entirety]. For assays, frozen HCT-116 cells were thawed and grown in McCoy's 5A Modified Medium (Gibco) supplemented with 10% (v/v) FBS. Cells were sub-cultured once, and then cells in log phase growth were harvested by trypsinization. Trypsinized cells were seeded into 96-well plates (1,000 cells/well) and incubated overnight at 37° C. in the presence of 5% CO₂. Compounds 6, 7, lazarimide C and cladoniamide (10 mg/mL in DMSO) were sequentially diluted in culture media (2-fold dilution starting at 50 μg/mL) across a 96-well plate and 100 μL was transferred to the appropriate well in an assay plate. The plates were incubated at 37° C. for 3 days and then evaluated for viability using a crystal violet-based colorimetric assay [See Zivadinovic et al., “Membrane estrogen receptor-alpha levels in MCF-7 breast cancer cells predict cAMP and proliferation responses.” Breast Cancer Res. 7(1):R101-112 (2005)]. Cell viability was recorded based on the absorbance at 590 nm in compound treated well relative to DMSO control wells.

The IC₅₀s observed for cladoniamide A, lazarimide C, and compounds 6 and 7 were 40.4 nM, 25.8 μM, 11.6 nM and 8.4 nM, respectively.

### Example 4

### General Synthesis Strategy for the Compounds Disclosed Herein

A general synthesis of compounds of the invention is set forth in Scheme 1.

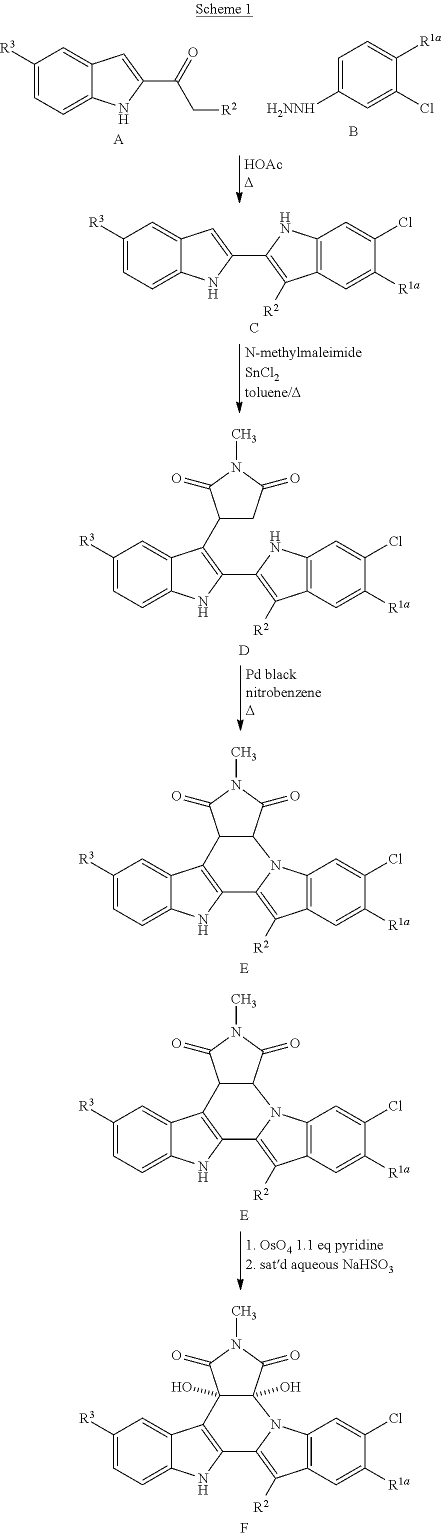

In Scheme 1, R^{1a} is hydrogen, halogen or a protected hydroxyl. When R^{1a} is a protected hydroxyl, a final step of deprotection is carried out, as described below, to provide R¹═OH.

A Specific Synthesis Follows:

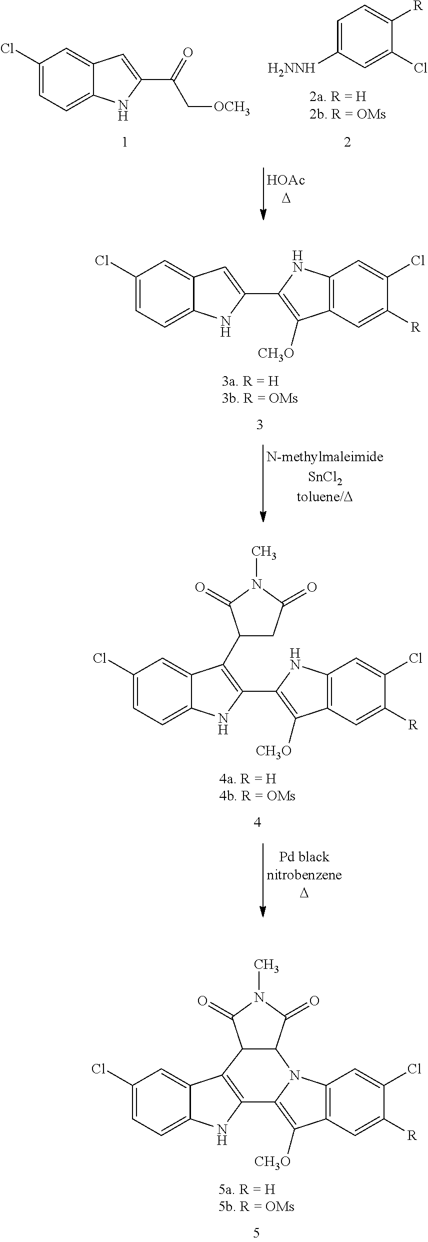

The synthesis of indolotryptolines 6a and 7 follows the procedure of Kimura et al. Organic Letters (2012), 14(17), 4418-4421. Acylindole 1, obtained by the method described in Kimura (op.cit), is reacted with two equivalents of hydrazine 2 in 10 mL of acetic acid per gram of 1 at reflux. The reaction is cooled, diluted with ethyl acetate and quenched with saturated aqueous sodium bicarbonate. The organic layer is washed with water, then saturated NaCl and dried over Na₂SO₄ and stripped to provide crude 3, which is used in the next step without purification.

The bisindole 3 is dissolved in toluene; 1.8 equivalents of N-methylmaleimide and a catalytic amount of SnCl₂ (0.16 equivalents) are added. The mixture is refluxed until reaction is substantially complete. The toluene is stripped off and the product 4 is purified by chromatography on silica gel with ethyl acetate/hexane.

The succinimidyl bisindole 4 is cyclized by treating with one equivalent of palladium black in nitrobenzene at elevated temperature (e.g. 200° C.). The reaction mixture is filtered through silica gel eluting with cyclohexane/chloroform to remove the nitrobenzene and then chloroform/methanol/trifluoroacetic acid to displace the product 5 from the silica. The eluate is concentrated, dissolved in ethyl acetate and washed with saturated aqueous NaHCO₃, then water, then brine. It is dried over Na₂SO₄, concentrated and passed through a silica gel column eluting with chloroform/methanol to provide pure 5.

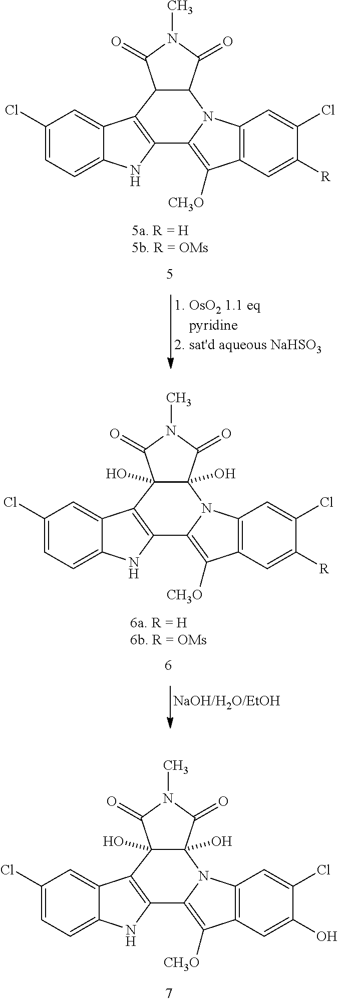

The fully assembled ring system 5 is dissolved in pyridine with one equivalent of osmium tetroxide and heated at 40° C. until starting material is consumed. Excess saturated aqueous NaHSO₃ is added and the mixture stirred vigorously for several hours at elevated temperature (e.g. 40-60° C.). The mixture is extracted into ethyl acetate and washed with water, then brine. It is dried over Na₂SO₄, concentrated and passed through a silica gel column eluting with chloroform/methanol to provide pure cis diol 6. Individual enantiomers of the cis diol may be obtained by chromatography on a chiral medium such as Daicel CHIRALPAC® IC using ethyl acetate/hexane.

For product 7, the cis diol 6b is dissolved in ethanol and treated with aqueous 2N NaOH. The ethanol is stripped off and the product extracted into ethyl acetate and washed with water, then brine. It is dried over Na₂SO₄ and the ethyl acetate is removed.

Substituted phenylhydrazine 2a (R═H) is available commercially from Aldrich. Substituted phenylhydrazine 2b is obtained by treatment of commercially available 2-chloro-4-nitrophenol with methansulfonyl chloride in the presence of base, followed reduction of the nitro (e.g. by hydrogenation over palladium catalyst in ethanol) and reaction of the aniline with sodium nitrite in the presence of aqueous acid (e.g. HCl or H₂SO₄).

Lazarimide A (7) was initially isolated from the extract of S. albus harboring the re-engineered Lzr gene cluster with four promoter cassettes (P1-P4) and the abeX1 oxidative gene, as described above. A pseudo molecular ion observed at m/z 486.0260 ([M-H]—) in the HRESIMS spectrum supports a molecular formula C₂₂H₁₅Cl₂N₃O₆. The HRESIMS spectrum displayed strong M+2 and M+4 signals, indicating the presence of two chlorines. As expected from bioinformatic analysis, the ¹NMR spectrum of 7 showed signals characteristic for the indolotryptoline family of compounds including a characteristic indole NH (H-13) at 11.59 ppm, an N-Me (H-14) at 2.86 ppm and aromatic signals around 8 ppm. Analysis of the COSY and HMBC spectra established two substructures designated as fragment a and fragment b. COSY correlations between H-1 (δH 7.51) and H-2 (δH 7.17, ortho coupling, J=8.6 Hz), and between H-2 and H-4 (δH 7.88, meta coupling, J=1.7 Hz) together with HMBC correlations from H-13 (δH 11.59, NH) to C-13a (δC 136.6) and C-12b (δC127.9), from H-1 (δH 7.51) to C-3 (δC 124.7) and C-4a (δC 126.0), from H-2 (δH 7.17) to C-4 (δC 119.4) and C-13a (δC 136.6), and from H-4 to C-4a (δC 126.0) and C-13a (δC 136.6) indicated the presence of an indole moiety with chlorination at C-3 and additional di-substitutions at C-4b and C-12b, which is typically observed in the indolotryptoline family of compounds. HMBC correlations from H-14 (δH 2.86) to C-5 (δC 174.2) and C-7 (δC 171.4), from C4c-OH (δH 7.16) to C-4b (δC 103.8), C-4c (δC 74.8), C-7a (δC 87.1) and C-5 (δC 174.2), and from C7a-OH (δH 8.17) to C-4c (δC 74.8), C-7a (δC 87.1) and C-7 (δC 171.4) revealed the structure of an N-methyl-dihydroxy succinimide with additional substitutions at C-4c and C-7a. This succinimide moiety was connected to a chlorinated indole moiety via an HMBC correlation from C4c-OH (δH 7.16) and H-4 (δH 7.88) to C-4b (δC 103.8) establishing substructure a. Substructure b was also defined by HMBC correlations. HMBC correlations from C10-O (δH 9.81) to C-9 (δC 118.0), C-10 (δC 147.2) and C-11 (δC 102.5), from H-8 (δH 8.04) to C-10 (δH 147.2) and C-11a (δC 121.2), from H-11 (δH 7.16) to C-7c (δC 130.4) and C-9 (δC 118.0), and from 12-OMe (δH 4.02) and H-11 (δH 7.16) to C-12 (δC 135.7) indicate that substructure b is another indole moiety with chlorination at C-9, hydroxylation at C-10, and methoxylation at C-12 as well as two additional substitutions.

HMBC correlations connecting substructures a and b were not observed due to the presence of quaternary carbons throughout the structure. However, the presence of the indolotryptoline scaffold could be deduced based on carbon chemical shift comparisons to known indolotryptolines, NOESY correlation data and the bioinformatics analysis of the gene cluster. The downfield-shifted chemical shift of C-7a (δC 87.1) compared to that of C4c (δC 74.8) suggests that C-7a is nitrogen-substituted. The presence of an indolotryptoline structure was further confirmed by an NOE correlation observed between 12-OMe and H-13 (NH). The NOE correlation observed between C4c-OH and C7a-OH suggested the “cis” configuration between these two hydroxyl groups, completing the structure determination of 7. The numbering system used in the foregoing discussion is as shown:

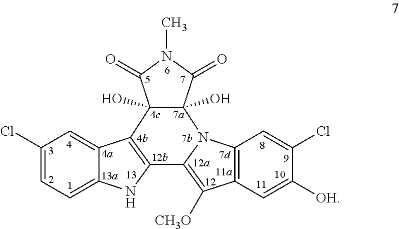

This is somewhat different from the numbering system used by Chemical Abstracts, which assigns the following numbering in the indexing of the indolotryptoline alkaloids:

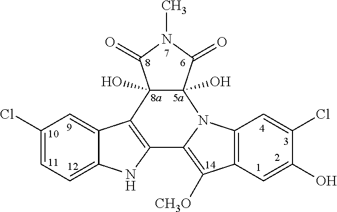

In the claims below, for internal consistency, we have used the 4c-7a numbering, which corresponds to Chemical Abstracts' numbering of 8a-5a as the corresponding chiral carbons.

Based on the X-ray crystal structure of lazarimide C,

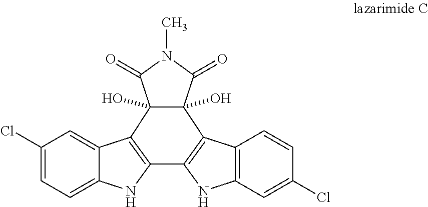 [SMILES: CN1C(=O)[C@@]2(O)c3c([nH]c4cc(Cl)ccc34)-c3[nH]c4ccc(Cl)cc4c3[C@@]2(O)C1=O]

the absolute configurations of hydroxyl group-bearing stereogenic carbons C-4c and C-7a in the structure of 7 can be deduced. The X-ray crystallography of lazaramide C assigned the configuration of C-4c as “S”, and the absolute configuration of this carbon is likely to remain the same during the oxidation-mediated indole ring flipping step, thus together with the “cis” relationship observed between hydroxyl groups at C-4c and C-7a, the absolute configurations of C-4c and C-7a in the structure of 7 were deduced as “S” and “R”, respectively. This assignment is consistent with the absolute configuration reported for cladoniamide A.

Lazarimide B (6) was isolated from the extract of S. albus harboring the reengineered Lzr gene cluster with three promoter cassettes (P1-P3) and the abeX1 oxidative gene. The molecular ion observed at 470.0320 ([M-H]—) in the HRESIMS spectrum indicates a molecular formula of C₂₂H₁₅Cl₂N₃O₅. The HRESIMS spectrum of 6 also showed strong signals corresponding to M+2 and M+4 indicating the presence of two chlorines. The structure of lazarimide B (6) was determined by comparison of the NMR spectra with those for compound 7. Compound 6 showed nearly identical ¹H and ¹³C NMR data to those of 7 except for the set of chemical shifts that would be predicted to correspond to the flipped indole ring moiety. In the ¹H proton spectra, differences in both chemical shifts and splitting patterns were observed. First, one additional proton signal was observed, which showed COSY correlations with H-8 (δH 8.11, J=1.8 Hz) and H-11 (δH 7.75, J=8.5 Hz). This, together with the fact that compound 6 was obtained without a promoter replacement for the operon containing the P450 hydroxylase gene, indicated that compound 6 is the non-hydroxylated analog of 7.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A compound of Formula I: 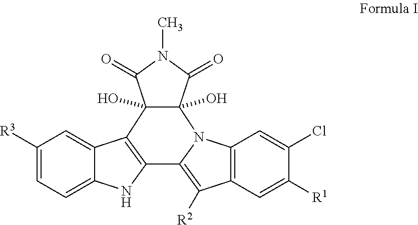
wherein
R¹ is chosen from hydrogen, halogen and OH;
R² is (C₁-C₄)alkoxy; and
R³ is chosen from hydrogen and halogen.

2. A compound according to claim 1 wherein R³ is chlorine.

3. A compound according to claim 1 wherein R² is methoxy.

4. A compound according to claim 1 wherein R¹ is hydrogen or OH.

5. A compound according to claim 4 wherein R² is methoxy.

6. A compound according to claim 5 wherein R³ is chlorine.

7. The compound 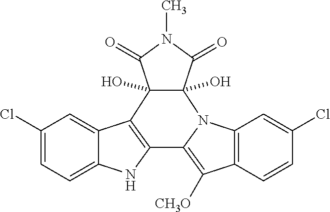
according to claim 1.

8. The compound 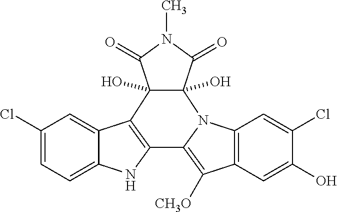
according to claim 1.

9. The (4cS,7aR) enantiomer 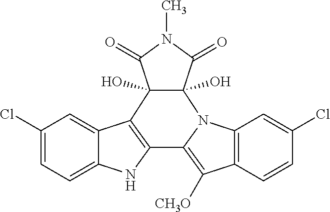
according to claim 7.

10. The (4cS,7aR) enantiomer 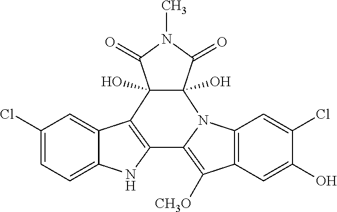
according to claim 8.

11. A pharmaceutical composition comprising a pharmaceutically acceptable carrier and a compound according to claim 1.

12. A method of treating an EGFR-overexpressing cancer comprising administering a compound according claim 1.

13. A method according to claim 12 wherein said cancer is chosen from head and neck, ovarian, cervical, bladder, renal and oesophageal cancers, non-small cell lung cancer, bronchoalveolar carcinoma, gastric, breast, endometrial and colorectal cancers.

14. A method according to claim 13 wherein said cancer is chosen from non-small cell lung cancer, refractory breast cancer, renal cancer and colon cancer.
